(19) United States
(12) Patent Application Publication
(10) Pub. No.: US 2007/0118044 A1
(43) Pub. Date: May 24, 2007

(54) Method and device for identifying; measuring and analyzing abnormal neurological responses

(75) Inventors: Arto Remes, Kuopio (FI); Pekka Tolvanen, Kuopio (FI); Juha Kylliainen, Siilinjarvi (FI); Markku Kankaanpaa, Kuopio (FI)

Correspondence Address: HARRINGTON & SMITH, PC, 4 RESEARCH DRIVE, SHELTON, CT, 06484-6212, US

(73) Assignee: Mega Elektroniikka Oy

(21) Appl. No.: 11/606,418

(22) Filed: Nov. 30, 2006

Related U.S. Application Data
(63) Continuation of application No. PCT/FI05/00330, filed on Jul. 18, 2005.

(51) Int. Cl. A61B 5/04

(52) U.S. Cl. 600/546; 128/905

(57) ABSTRACT

The objective of the invention is a method and a device for identifying, measuring and analyzing abnormal neurological responses. In the method in accordance with the invention neurological diseases, pain, fatigue, power producing ability and/or activeness and/or symptoms connected with those are estimated by measuring signals produced by muscles and by analyzing signals with nonlinear calculation methods. The device in accordance with the invention includes sensors to be placed in the body or on the body surface for measuring signals produced by muscles, a signal-analyzing device for analyzing signals with nonlinear calculation methods and a feedback device for giving feedback.

The present invention relates to a analysis method for diagnosis, treatment, follow-up of pain and/or other central nervous system related diseases, objective measurement ment of fatigue and force production ability, and monitoring of changes in the vigour level of central nervous system.

Electric signal of muscles i.e. Electromyography (EMG) measured from the skin surface describes activation and functioning of muscle cells/motor units during the contraction of a skeletal muscle. It is commonly known that by means of an EMG-signal it is possible to measure activity levels of muscles and that from those calculate various quantities describing the functioning of muscles and the body. EMG-signal is usually measured through electrodes placed on the skin overlying muscles.

As muscle power increases, the central nervous system recruits in increasing amount muscle cells/motor units in order the muscle power to achieve the functional level to enable the growing mechanical performance. The accuracy of a mechanical performance and the power supply of a muscle function are under a complex response system, in which besides the central nervous system also the gongi tendon organs and muscle bundles with their response systems take part.

Experience of muscles and the size of muscle cells, as well as activeness of the central nervous system have influence on the muscle power supply. The energy transform of a muscle (e.g. the function of mitochondria) and the distribution of muscle cell types, which depend on the degree of mechanic experience of the muscle, have effect for their part on endurance property of muscle cells.

As a performance lasts longer and the skeletal muscle gets tired, its function changes in accordance with the energy transform and the muscle cell type/distribution (fast-twitch/slow-twitch). When tired the conduction velocity of action potential gets slower on the membrane of a muscle cell along with saturation of K+ and Na+ ion change and recruiting getting slower. In a powerful performance, leading to fatigue of the skeletal muscle the oxygen intake to energy production weakens and the cell acidifies as the result of accumulation of H+ ion concentration inside the cell. As the result the velocity of action potential wave on the muscle membrane gets slower being longer in time (=frequency gets lower).

Fatigue of a muscle is in accordance with current knowledge also the result of saturation of recruiting processes and response systems on central nervous system level. On central nervous system level as a muscle gets tired, the recruiting of muscle cells changes as the result of the influence of changes in function of the central nervous system and the response systems, in which case the function of motor units in the muscle synchronizes. As the result of this, the measured EMG-signal changes to be less complex and its frequency gets lower.

Typically, the EMG- signal of a healthy person is stochastic and even nonstationary /chaotic in some respect. In abnormal situations (e.g. pain, a disease of the central nervous system, Parkinson's disease, lowering of activeness, chronical fatigue, depression) there occurs changes of motor control and its response systems on central nervous system level, which changes lead to decreasing of complexity of the EMG-signal. In diseases on central nervous system level, there are typically changes in the functions of neurotransmitters of the brain (e.g. dopamine in Parkinson's disease and serotonin in depression) which partly change the motor firing system and also affect the functions of response systems disadvantageously.

EMG-signal is usually measured in two different ways depending on what kind of measuring accuracy or analysis of results are wanted or, on the other hand, what kind is the technical operating capacity of measuring equipment. The most versatile measuring and analyzing options are achieved by measuring EMG bi-polar as so-called RAW EMG-signal. By utilizing sufficiently high sampling frequency, for example 1000-10000 Hz it is possible to make analysis from a signal, precise enough for scientific research by means of FFT spectrum calculation. Equipment, measuring RAW signal has to possess great capacity of processing and great memory capacity. Another typical way of measuring is so-called averaging i.e. AVERAGE-EMG method, in which method the EMG-signal received from a muscle is first rectified and after that averaged sliding across averaging time chosen for the purpose, for example 0,1-1 seconds. Typically, loading of muscles is estimated, loading comparisons are made, activating rates and times of muscles are examined and so on by analyzing the AVERAGE-EMG-signal. Measuring of AVERAGE EMG-signal may in principle be made with a rather simple equipment.

Each EMG-signal forms a so-called profile, in which active and passive states of the measured muscle may be seen as changes in amplitude of the signal. In addition, the frequency spectrum of the signal contains information on the function of the muscle. Quantities describing the function of muscles among others are:
muscle load and changes in it
distribution of load on different muscles of the body
the side-difference between two symmetrical muscles, when loading simultaneously with the same load
activation order, activation times, reaction times and so on of muscles
comparison of activation profiles of muscles in various states of a performance and on different test persons
fatigue of muscles

In addition, from the shape, amplitude, frequency spectrum and changes in those of an EMG-signal it is possible to calculate plenty of other quantities to be monitored and controlled. The measured EMG-signals are transmitted typically either to be presented in real-time on a display device or signals are stored in file format for later examination and analyses.

It is commonly known that the performance level and the degree of fatigueness may be estimated by measuring and analyzing an EMG-signal produced by a muscle. One method of this kind has been described in U.S. Pat.5,361,775 by Mega Elektroniikka Oy, among other things, in which by a FFT—i.e. spectrum analysis made from a EMG-signal several quantities correlating with fatigue of a muscle are able to be received. Fatigue of a muscle may be seen in power spectrum of an EMG-signal among other things as lowering of mean-frequency and, on the other hand, as rising of averaged EMG-activity level.

Linear methods such as FFT-analysis may be applied limited for analysing of an EMG-signal while a linear analysis of an unstationar and stochastic signal averages the result and therefore a part of the real information from the signal is lost. Nonlinear methods (such as complexity analysis) are more applicabable for analysis of stochastic EMG-signal. These kinds of algorithms are e.g. entropy, determinism, complexity-algorithms. They are proved to be more applicabable than linear algorithms for analyzing biosignals and therefore their use applied for diagnostic use and monitoring treatment of diseases is essentially better manner

Down to the stochastic and non-stationary character of signals a nonlinear analysis is more applicabable than a linear analysis for analysing of biosignals. It is easier to get information from pain response and motoric and premotoric states of operation model interferences and changes in cerebellum/corpus callosum level, among other things. In that case weakening of transmitters such as dopamine and serotonine levels interacts to complexity of EMG-signals measured from a muscle, and so called fatigue changes are more powerful than those of a normal group.

A nonlinear method used for analysing of biosignals is so called Lempel-Ziv-complexity analysis C(n) which searches periodicities and regularities existing in an EMG-signal.

Another frequently used method is so-called RQA i.e. Recurrence Quantification Analysis, which has been developed especially for studying nonlinear systems. Eckmann was the fist to publish RQA in 1987 and Webber introduced it in physiological studies in 1994. As RQA method is not dependent e.g. on the quantity of data or stationarity or statistical distribution of data content, RQA is well applicabable in physiological systems, to which sudden changes, variations of levels, noise and so on originating in living organisms are often related. For example, in measuring fatigue the RQA method detects the starting moment faster and more sensitively than the FFT method. However, these analyses have not been used in disease grading in diagnostic sense.

The objective of the invention is to provide a method for diagnostics of pain and Parkinson disease and/or other diagnostics, for follow-up of Parkinson disease treating, for objective measurement of muscle fatigue, and in generally for follow-up of the vigour level changes of central nervous system, of which method these matters/diseases is possible to identify easier, more objective and better than with present diagnostic methods.

In the analysis method according to invention nonlinear analysis method is applied with the help of application of measurement system for diagnostics of pain and Parkinson disease, follow-up of Parkinson disease treatment, objective measurement of muscle fatigue, and in general for changes in vigor level of central nervous system, in which method parameters relating to aforementioned symptoms and diseases is calculated from the EMG-signals obtained from muscles by using non-linear methods, and when necessary database is compared to collected reference values or former own values for concluding the progression of disease, pain state or aforementioned application discovery from changes and deviations.

The nonlinear estimation method application of an EMG-signal enables e.g. following ing applications:
a) Objective estimation of pain
b) Estimation of decreasing of dopamine level of the brain typical of Parkinson's disease, which enables screening of persons in a risk group of Parkinson's disease (identifying of prestages), diagnosing of the disease and the follow-up of progression of the disease
c) By means of the method it is possible to time and dose the medical treatment of a patient of Parkinson's disease
d) Objective measuring of muscle fatigue
e) Follow-up of changes in activeness of the central nervous system due to various reasons (e.g. dementia, the Alzheimer disease, depression, chronical/acute fatigue)
f) Follow-up of development of motoric skills of the body and parts of it

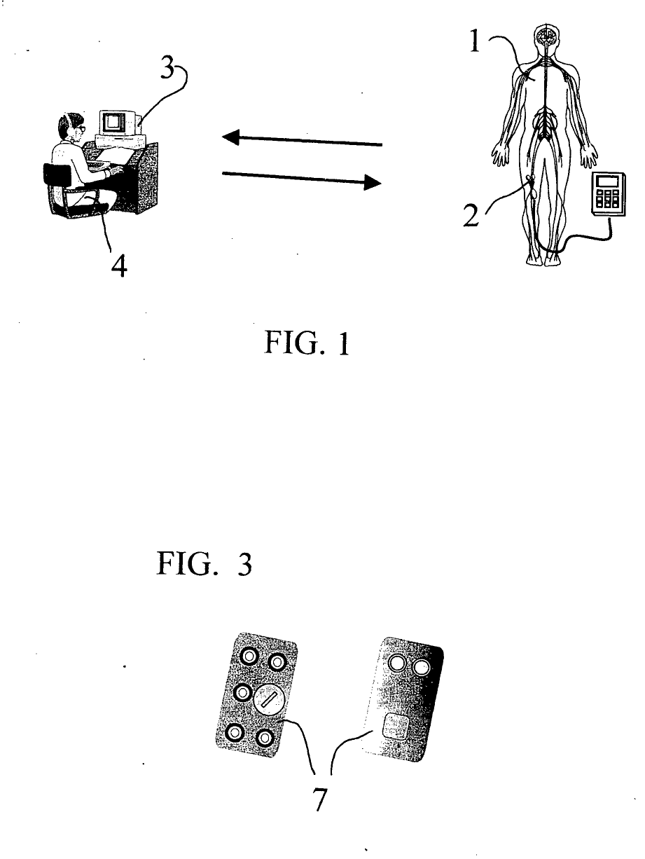

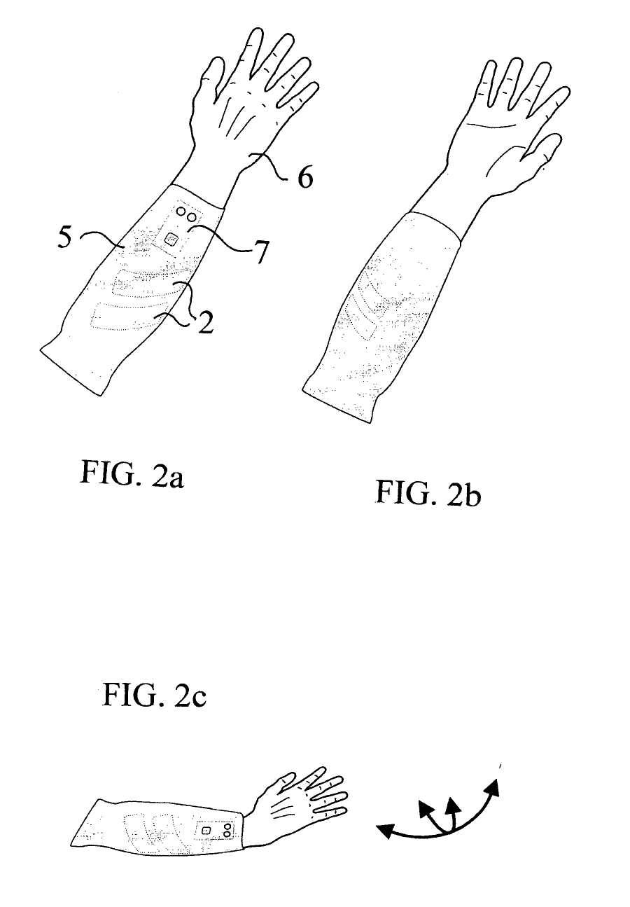

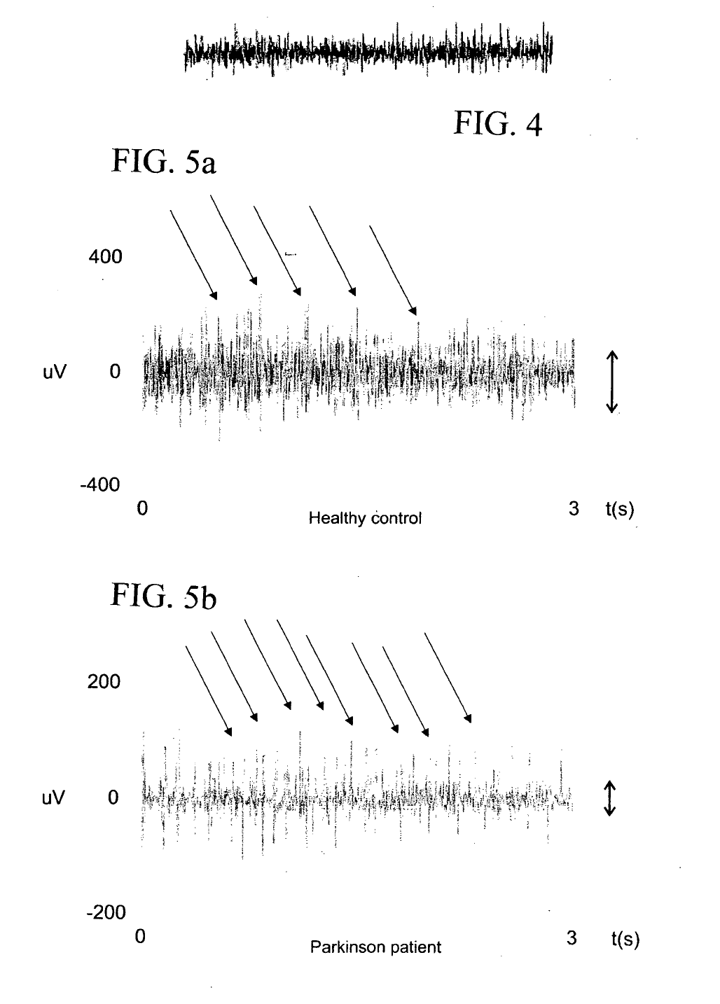

FIG. 1 illustrates different stages of a method in accordance with invention.

In an advantageous application of the invention pain response experienced by patients suffering from back pain in comparison with feelings of patients with similar by performance ability of muscles but with no pain. Condition of back muscles of patients with back problems has weakened and a part of patients suffers from pain also while loading muscles. Patients with back pain may be separated from healthy patients who have been through treatment/training by means of FFT method based on linear analysis, but the method does not separate individually pain patients. Identification of pain patients is successful while using the nonlinear RQA method and furthermore the method makes it possible to make a measurement from a measuring signal before rehabilitating exercise part and after it, whereas earlier the measurement had to be carried out with a linear method during the whole performance.

In second advantageous application of the invention, the method is utilized in diagnosing and treating of Parkinson's disease. Patients with Parkinson's disease suffer from changes in motoric control and its response system on the central nervous system level, which changes are caused by changes of dopamine level. These changes may be seen as decreasing of complexity of the EMG-signal, which may be detected by a nonlinear analysis such as e.g. RQA. Based on this the follow-up of

Parkinson's disease patients or risk groups may be realized before outbreak of the disease.

The diagnosis method of Parkinson's disease is based on measuring made on a test person with an EMG-equipment and comparing of measuring results to comparison data in a database. In this context FIG. 1 is referred, in which the different stages of the method is illustrated. Typically, an EMG-measurement is carried out on the muscles of the arm as the motoric control of arms is the most developed form of neuromuscular function. EMG-signal samples are taken with various standard motion course and power tests, in which case comparison of results with the database is possible. The motion courses to be tested are e.g. removing of an object of certain size and weight from one place to another along defined course. Along the course, the arm and the wrist must simultaneously e.g. clasp the object, move in horizontal and vertical directions as well as twist from one extreme position to another. In addition, the spontaneous EMG-responses of the muscle may also be analyzed and separate the healthy and the Parkinson patients on grounds of the measuring data by a normal every day muscle activation analysis.

The comparison database contains sample data both from the healthy and from the patients in various phases of Parkinson's disease. In this case, the EMG-sample from a test person may give suggestions not only about the first symptoms of the disease but also about the proceeding degree of the disease. The results are normalized on grounds of data base comparison such that the proceeding degree may be categorized with a suitable scale.

The method may also be applied in dosing of medical treatment of Parkinson's disease. The patient has an automatic dosing apparatus adjusted, which e.g. infuses medicine to the blood circulation or to the small intestine of the patient by means of estimation of the dopamine level based on EMG-measuring. EMG-measuring is made, for example, by means of a measuring wristband, which is easy to use and continuously functioning. A measuring wristband or other sensor is placed, for example, on the hand, feet and so on. Response from the measuring signal is analyzed at regular time intervals, for example every 5 min. When in the EMG-response, the analysis of which has been carried out by means of some nonlinear algorithms mentioned, has visible signs of falling of the dopamine level, the information is transmitted to the device the patient is carrying. The device may be a dosing pump or some other dozer of medicine, which has e.g. an alarm system for starting the dosing or for a dose of medicine to be taken internally. In more advanced models, syringing may start automatically through an invasive canule. It is also characteristic to Parkinson's disease that the symptoms appear first on one limb, in which case by comparing signals both on the left and the right limb the ending of the influence of medicine may be anticipated in good time.

In another advantageous application of the invention, the method is utilized in measuring of fatigue during dynamic motion. In the patent U.S.5,361,775 of Mega Elektroniikka Oy a FFT analysis made from so-called RAW EMG-signal is described, by means of which analysis quantities correlating with muscle fatigue are calculated. The method has been proved fucntionable while measuring muscle fatigue during static load. FFT calculation gives suggestive results also during dynamic motion but muscle contractions varying continuously and interferences due to motion change the frequency content of the EMG-signal such that the results from FFT calculations are not as precise and reliable as in static measurement. FFT calculation made of dynamic motion may be specified by making use of motion sensors attached to a person during the test, by means of which sensors the interferences created in signals may be eliminated or limit the areas included in FFT calculation to advantageous in terms of calculation. In this case fatigue may be measured reliably e.g. during work or sports performance with a measuring/displaying device to be carried with a person.

In third advantageous application of the invention, the method is utilized for measuring the activeness level of a person which is necessary for follow-up of changes in activeness state of the central nervous system due to various reasons e.g. in connection with diseases such as dementia, Alzheimer disease, depression, chronic/acute fatigue and so on. Control of the state of fatigueness is useful also in some working positions requiring continuous concentrating such as surgery, flying/air control and so on.

In this application, the median value of a person is defined for complexity, which is compared to reference data. The person to be measured carries a measuring device, which measures at regular time intervals e.g. EMG-signal from the muscles of the arms, from which signal some of the parameters complexity, entropy or determinism is calculated. From the value, e.g. the mean value of a day or a week is calculated. The change with respect to the mean value on a long time period describes the change in activeness, variation in serotonin level, motoric learning and so on.

An advantageous application of the invention measures development of motoric skills of a person. The method is utilized e.g. in rehabilitation after a person has been disabled, in training of professions requiring great accuracy or e.g. in sports training. EMG-signal is measured from one or several muscles during a performance. During the measurement a specific performance and its training part are aimed to be specified e.g. a golf swing or a tennis serve. The measuring sensors are placed on the skin overlying muscles participating in the performance at the middle of the muscle. The measuring is carried out through disposable sensors or through sensors integrated in clothing. The information of measuring is stored in a measuring device on the wrist, on the waist or in the pocket, which measures the EMG-signal with recognized methods. The measuring signal is calculated by means of various calculation algorithms, by calculating e.g. complexity values during the performance. The performance is tried to be separated into various phases and the result of the performance is fed also into the memory of the device. Complexity values from the entire performance are calculated and stored into the memory. As the motoric level develops the signal of complexity changes and the time-related synchronisation of muscles during a performance improves. The change is compared to the received performance results. Later on, the values may be utilized while restoring and further developing motoric skills. The results may be compared to the values of the database and in this way estimate the development of motoric learning and skill objectively.

1. Method for identifying, measuring and analyzing abnormal neurological responses, in which
neurological diseases, pain, fatigue, power producing ability and/or activeness and/or symptoms connected with those are estimated by measuring signals produced by muscles and by analyzing signals with linear and/or nonlinear calculation methods and by measuring and analysing signals produced by body or limb motions.

2. Method in accordance with claim 1, in which
electrical signals of muscles are measured with a measuring device.

3. Method in accordance with claim 1, in which
electrical signals of active muscles are measured with a measuring device.

4. Method in accordance with claim 1, in which
results received by the method are compared to reference results stored in a database.

5. Method in accordance with claim 1, in which
results received by the method are transmitted to a user by means of a feedback device.

6. Method in accordance with claim 1, in which
received results are transmitted to the person examined by means of a personal feedback device.

7. Method in accordance with claim 1, in which
Parkinson's disease is diagnosized, the influence of rate of dosage of medicine is defined, monitored and controlled and the progression of the disease is followed by the method.

8. Method in accordance with claim 1, in which
pain is diagnosized, the influence of rate of dosage of medicine is defined, monitored and controlled and the influence of pain is followed by the method.

9. Method in accordance with claim 1, in which
the degree of fatigueness is measured by the method.

10. Method in accordance with claim 1, in which
power producing ability and changes in it are measured by the method.

11. Method in accordance with claim 1, in which
activeness of the central nervous system and changes in it are measured by the method.

12. Device for identifying, measuring and analyzing abnormal neurological responses, comprising
sensors to be placed in the body or on the body surface for measuring signals produced by muscles, a signal analyzing device for analyzing signals with linear and/or nonlinear calculation methods and a feedback device for giving feedback.

13. Device in accordance with claim 12, comprising
sensors to be placed in the body or on the body surface for measuring myoelectrical signals produced by neuromuscular system.

14. Device in accordance with claim 12, comprising
sensors to be placed in the body or on the body surface for measuring movement or acceleration signals produced by muscle and limbs.
